(19) United States
(12) Patent Application Publication
(10) Pub. No.: US 2013/0084322 A1
(43) Pub. Date: Apr. 4, 2013

(54) DRUG-IMPREGNATED BIODEGRADABLE STENT AND METHODS OF MAKING THE SAME

(71) Applicant: Tim Wu, Shrewsbury, MA (US)

(72) Inventor: Tim Wu, Shrewsbury, MA (US)

(73) Assignee: Tim Wu, Shrewsbury, MA (US)

(21) Appl. No.: 13/685,969

(22) Filed: Nov. 27, 2012

Related U.S. Application Data
(63) Continuation of application No. 13/330,637, filed on Dec. 19, 2011, now abandoned.
Application No. 13/330,637 is a continuation-in-part of application No. 13/014,750, filed on Jan. 27, 2011.
(60) Provisional application No. 61/427,141, filed on Dec. 24, 2010.
(60) Provisional application No. 61/368,833, filed on Jul. 29, 2010.

(51) Int. Cl. A61F 2/82

(52) U.S. Cl. CPC A61F 2/82
USPC 424/426; 514/449

(57) ABSTRACT

The present invention relates to a drug-impregnated implantable medical device such as stent manufactured from polymers, and more particularly, biodegradable polymers including biodegradable polyesters. The invented medical devices include at least one therapeutic agent impregnated in at least one biodegradable polymer wherein at least a portion of the therapeutic agent in this polymer is crystalline. The device and methods to impregnated one or more therapeutic agents, where each therapeutics agent may be chosen from the following categories: immunosuppressant agents, anti-neoplastic agents and anti-inflammatory agents were disclosed. Other embodiments include methods of fabricating drug-impregnated implantable medical devices.

### CROSS-REFERENCE TO RELATED APPLICATIONS

This application is the continuation-in-part of the U.S. patent application Ser. No. 13/330,637, filed on Dec. 19, 2011 which claims the benefit of the U.S. provisional application No. 61/427,141, filed on Dec. 24, 2010. This application is also a continuation-in-part of the U.S. patent application Ser. No. 12/209,104, filed on Sep. 11, 2008, the U.S. patent application Ser. No. 11/843,528, filed on Aug. 22, 2007 and U.S. patent application Ser. No. 13/014,750 filed on Jan. 21, 2011. The disclosures of all of which are hereby incorporated by reference in their entireties.

### FIELD OF THE INVENTION

The present invention relates to a biodegradable drug-eluting stent comprising at least one therapeutic agent encapsulated inside at least one biodegradable polymer wherein the encapsulated therapeutic agent would be sustainably and controlled released.

The present invention encompasses the discovery that at least one therapeutic agent can be encapsulated into at least one biocompatible polymer through extrusion or injection molding process to form solid tubular structure for subsequent drug-eluting stent fabrication, and at least a portion of the encapsulated therapeutic agent in such drug-containing polymeric tube is crystalline.

The present invention further provides the methods of fabricating drug-containing implantable biodegradable medical device such as stent that effectively controls sustained release of the anti-neoplastic agent and the immunosuppressant agent. The present invention also encompasses the finding that medical devices encapsulated with such drug or a drug-combination are surprisingly effective in inhibiting, preventing, and/or delaying the onset of hyper proliferative conditions such as restenosis in vivo. The present invention therefore provides, among other things, a drug-containing implantable medical device comprising an immunosuppressant agent, an anti-neoplastic agent encapsulated in at least one biocompatible polymers. The present invention further provides medical devices encapsulated with at least one therapeutic agent according to the invention and other drug delivery or eluting systems and methods of their uses.

In one aspect, the present invention related to a drug-containing implantable medical device comprising at least one therapeutic agent were encapsulated inside at least one biocompatible polymer, wherein the drugs are characterized with sustained-release of the immunosuppressant agent, an anti-neoplastic agent, and an combination of both for at least about 4 weeks (e.g., at least 5 weeks, 6 weeks, 7 weeks, 8 weeks, 9 weeks, 10 weeks, 11 weeks, 12 weeks, or longer).

In some embodiments, suitable immunosuppressant agent is sirolimus or a prodrug or analog thereof. In some embodiments, suitable immunosuppressant agents are selected from zotarolimus, tacrolimus, everolimus, biolimus, pimecrolimus, supralimus, temsirolimus, TAFA 93, invamycin, neuroimmunophilins, or combinations or analogs thereof. In some embodiments, suitable anti-neoplastic agent is paclitaxel or a prodrug or analog thereof. In some embodiments, suitable anti-neoplastic agent is selected from carboplatin, vinorelbine, doxorubicin, gemcitabine, actinomycin-D, eisplatin, camptothecin, 5-fluorouracil, cyclophosphamide, 1-β-D-arabinofuranosylcytosine, or combinations or analogs thereof.

In some embodiments, therapeutic agent encapusulated inside the biocompatible polymer in accordance with the invention further include one or more anti-thrombotic agents, anti-proliferative agents, anti-inflammatory agents, anti-migratory agents, agents affecting extracellular matrix production and organization, anti-mitotic agents, anesthetic agents, anti-coagulant agents, vascular cell growth promoters, vascular cell growth inhibitors, cholesterol-lowering agents, vasodilating agents, and/or agents that interfere with endogenous vasoactive mechanisms. For example, in some embodiment, the combination of immunosuppressant and anti-neoplastic in a ratio, by weight, ranging from about 1:99 to 99:1. (e.g., 10:90, 20:80, 30:70, 40:60, 50:50, 60:40, 70:30, 80:20, 90:10). In some embodiments, the anti-neoplastic agent and immunosuppressant agent are present in a ratio by weight of approximately 1:1 (i.e., 50:50). In some embodiments, the anti-neoplastic agent and immunosuppressant agent are present in an amount ranging from about 0.1 μg/mm² to about 5 μg/mm² (e.g., 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, μg/mm²).

In some embodiments, polymers suitable for the present invention contains a biodegradable polymer. In some embodiments, the biodegradable polymer is a polyester polymer. In some embodiments, suitable polyester polymer include, but are not limited to, poly(D,L-lactide-co-glycolide) (PLGA), polylactide (PLA), poly(L-lactide) (PLLA), poly(D,L-lactide (PDLA), polyglycolides (PGA), poly(D,L-glycolide) (PLG), and combinations thereof. In some embodiments, polymer in accordance with the invention further contains a calcium phosphate. In some embodiments, suitable calcium phosphates include, but are not limited to, amorphous calcium phosphate (ACP), dicalcium phosphate (PCP), tricalcium phosphate (TCP), pentacalcium hydroxyapatite (HAp), tetracalcium phosphate monoxide (TTCP), and combinations thereof. In some embodiments, the biodegradable polymer and calcium phosphate are present in a ratio (by weight) of about 1:99 to 99:1 (e.g., 10:90, 20:80, 30:70, 40:60, 50:50, 60:40, 70:30, 80:20, 90:10).

In some embodiments, polymers suitable for the present invention include a nonbiodegradable polymer. In some embodiments, suitable nonbiodegradable polymers include, but are not limited to, poly-n-butyl methacrylate (PBMA), polyethylene-co-vinyl acetate (PEVA), poly (styrene-b-isobutylene-b-styrene) (SIBS), and combinations thereof.

In some embodiments, the immunosuppressant agent and anti-neoplastic agent and anti-thrombotic are present in the same layer. In some embodiments, the immunosuppressant agent and anti-neoplastic agent and anti-thrombotic agent are present in different layers.

In another aspect, the present invention provides methods for fabricating drug-containing implantable medical device, more specifically, a drug-containing biodegradable drug-eluting stent, including drug-polymeric composition compounding, drug-containing polymeric composition tube forming, polymeric and drug molecular orientation, stent laser cutting etc. In some embodiments, the compoundable polymer and drugs are crystallized by various nanotechnologies and the drug-containing tube is then extruded through an extruder or injection molding with the drug-polymeric composition at the temperature of equal or above polymer melting point but below the encapsulated drug's melting point. In one embodiment, the nanoparticle sized polymer and drug are premixed before extrusion or molding and be extruded to solidified drug-containing tubular structure through extruder under the temperature between the polymer and drug's melting point. In another embodiment, the nanoparticle-sized/crystallized drugs are added to the molten polymer through a downstream feeder in an extruder. In an preferred embodiment, two or more therapeutic agents are be add in either the same layer of polymer or in the deferent layer of the tube through multiple layer extrusion technology.

In some embodiment, the formed tubes are further deformed radially and axially to orientate both the polymer and drug molecule direction with the blow molding technology to increase the tube's mechanic strength and drug's crystalinity. The deformed tubes are then subjected to laser cutting which is a know art according to the stent design pattern.

### BACKGROUND OF THE INVENTION

Coronary Artery Disease (CAD) has been the number one killer in the United States since 1900 and still remains the most common cause of death in the Western world despite therapeutic advances. Drug-Eluting Stent (DES) is currently the major therapy for CAD treatment. DES not only increases procedural success rates, but also increases the safety of procedures by decreasing the need for emergency coronary artery bypass graft surgery (CABG). As a result, stents are currently utilized in over 85% of the two million Percutaneous Coronary Intervention procedures (PCIs) in the US. The total direct cost for these life-saving procedures is over $2 billion annually. Despite the prevalent use of DES, there are significant drawbacks, including the need for costly, long-term anti-platelet therapy, as well as the metal artifact remaining in the vessel. Coronary stents are only required to provide scaffolding for up to six months following the procedure, however, since the stent remains in the vessel, potential long term complications may arise. In addition, the remaining, metal scaffolding precludes the vessel from returning to its natural state and prevents true endothelial repair and arterial remodeling. Following are brief descriptions of the two major issues existing in current DESs.

In-Stent Restenosis (ISR): ISR is the re-narrowing of an opened artery after stenting due primarily to the proliferative response of the intima, a layer of cells that line the lumen of the vessel, composed of connective tissue and smooth muscle cells (SMC). ISR has been the biggest problem in PCI until the recently successful development of DESs. Initially, the restenosis rate is as high as over 50% within six months post balloon dilation. Stenting lowers this number to 20-30%. DESs can significantly reduce the rate of restenosis to <10%. However, ISR in patients with high risk such as small vessels, diabetes, and long diffusion diseased arteries still remains unacceptably high (30%-60% in bare metal stents and 6%-18% to DESs).

Thrombosis: In spite of restenosis remaining a clinical problem in approximately 10% with DES implantation, it can often be successfully treated with repeated DES implantation. The greatest concern, however, has been of stent thrombosis which is associated with a high rate of myocardial infarction and death. The rate of early stent thrombosis (less than 30 days following implantation) appears similar in both bare metal stents (BMS) and DESs. However, late stent thrombosis (LST) has been increasingly reported beyond 12 months following DES implantation, with the greatest risk occurring as a result of premature discontinuation of antiplatelet therapy. Although the precise mechanism of late stage stent thrombosis is unknown, it is generally believed that the combination of delayed endothelialization due to antiproliferative therapy and persistence of the nonerodable polymer contribute to the hypersensitivity reaction, possibly with some residual active drug that may not be eluted.

Therefore, the challenges faced by emerging technologies are to reduce restenosis in high-risk lesions without compromising healing in order to avoid late thrombotic complications, and to improve system deliverability in order to allow the devices to treat more complex patients. Currently, a number of strategies are being utilized to achieve these goals, through the development of novel stent platforms, coating with biodegradable polymer or move away from polymers, and with new generations and/or combinations of biological agents that both inhibit proliferation and promote endothelialization. With the recent positive data from Abbott's ABSORB trial, clinical consensus is building that fully biodegradable stents (BDS) represent the next generation in DES.

Bioabsorbable and biodegradable materials for manufacturing temporary stents present a number of advantages. The conventional bioabsorbable or bioresorbable materials of the stents are selected to absorb or degrade over time to allow for subsequent interventional procedures such as restenting of the original site if there is restenosis and insertion of a vascular graft. Further, bioabsorbable and biodegradable stents allow for vascular remodeling, which is not possible with metal stents that tethers the arterial wall to a fixed geometry. In addition to the advantages of not having to surgically remove such stents, bioabsorbable and biodegradable materials tend to have excellent biocompatibility characteristics, especially in comparison to most conventionally used biocompatible metals. Another advantage of bioabsorbable and biodegradable stents is that the mechanical properties can be designed to substantially eliminate or reduce the stiffness and hardness that is often associated with metal stents, which can contribute to the propensity of a stent to damage a vessel or lumen. Examples of novel biodegradable stents include those found in U.S. Pat. No. 5,957,975, and U.S. application Ser. No. 10/508,739, which is herein incorporated by reference in its entirety.

However, in all current commercially available DESs and investigational biodegradable DESs, the drugs were coated on the stent surface in approximate 10 um thicknesses. These drug-containing polymeric compositions coated on stent surface are typically formed by dissolving one or more therapeutic agents and one or more biocompatible polymers in one or more solvents, followed by removing the solvents to form a solidified drug-containing polymeric composition. The solvent removal or solidification can be carried out using various techniques, including, but not limited to: spray drying (for preparation of coatings), solvent casting or spin coating (for preparation of thin films or membranes), and spinning (for preparation of fibers).

The solidified drug-containing coating compositions so formed typically contain the therapeutic agents in an amorphous phase. Amorphous therapeutic agents are very unstable, especially at temperatures that are above their glass transition temperatures. The amorphous therapeutic agents may gradually degrade over time, due to oxidation in the presence of oxygen. Such amorphous therapeutic agents can also become plasticized during device sterilization processes. Furthermore, therapeutic agent coated on the surface of medical device in this manner, are confined in or on the surface of the implantable medical devices amorphously by the biocompatible polymer and can be released into the surrounding environment in less than four weeks. As the restenosis forms in approximately 3 months and the impaired vascular remold process complete in approximately 6 months post stent implantation, the four weeks drug release period is theoretically neither longer enough for inhibiting restenosis formation nor for impaired vascular remolding, therefore there a need of a new drug-eluting stent with prolonged drug-release kinetics (at least over four wks) and improved drug-stability.

The present invention provides a biodegradable drug-eluting stent system comprising at least one therapeutic agent encapsulated inside biodegradable polymeric stent with controlled, sustainably release of therapeutic agent to the disease site. The invention, also provides the methods of fabricating the stent.

### SUMMARY OF THE INVENTION

In one aspect, the present invention include a bioabsorbable drug-eluting stent fabricated with a drug-containing polymeric composition wherein at least one therapeutic agent were encapsulated inside at least one biodegradable polymer, more specifically, biodegradable polyester polymer. Each encapsulated therapeutics agent is selected from the group consisting of immunosuppressant agents, anti-neoplastic agents and anti-thrombotic agents, and at least a portion of those encapsulated therapeutic agent in this polymer is crystalline.

In one aspect, the present invention include a bioabsorbable drug-eluting stent fabricated with a drug-containing polymeric composition wherein two or more therapeutic agent were encapsulated inside at least one biodegradable polymer, more specifically, biodegradable polyester polymer. Each encapsulated therapeutics agent is selected from the group consisting of immunosuppressant agents, anti-neoplastic agents and anti-thrombotic agents, and at least a portion of those encapsulated therapeutic agent in this polymer is crystalline.

In another aspect, the present invention includes a method of fabricating a biodegradable drug eluting stent with drug-containing polymeric composition. The method includes the following processing operations: drug and polymer precrystalization and drug-polymeric composition compounding with various nanotechnologies, drug-containing polymeric composition tube forming, polymeric and drug molecular orientation, stent laser cutting etc. The therapeutic agent is selected from the group consisting of immunosuppressant agents, anti-neoplastic agents, anti-thrombotic agent, wherein the at least one therapeutic agent is amorphous; Deforming the formed drug-containing tube would at least crystalline a portion of those encapsulated therapeutic agent in polymer.

Preferably, at least 10% of the therapeutic agent in the fabricated medical device of the present invention is crystalline. More preferably, at least 50% of the therapeutic agent in the medical device of the present invention is crystalline. Most preferably, at least 90%, 95%, or 98% of the therapeutic agent its the medical device is crystalline.

### BRIEF DESCRIPTION OF THE DRAWING

FIG. 1: illustration of an exemplary drug-implemented biodegradable stent of the invention.

FIG. 2: Exemplary illustrating HPLC analysis results of paclitaxel impregnated in the invented drug-impregnated biodegradable stent pre and post extrusion.

FIG. 3 depicts exemplary results of restenosis different between paclitaxel-impregnated PLLA and PLLA stents in pig coronary artery at one month post implantation.

FIG. 4 illustrates exemplary results of the histological changes (neointima and residual arterial lumen area) between paclitaxel-impregnated PLLA and PLLA stent groups at one month post imputation.

### DEFINITIONS

Agent: as used herein, the term “agent” refers to any substance that can be delivered to a tissue, cell, vessel, or subcellular locale. In some embodiments, the agent to be delivered, is a biologically active agent (bioactive agent), i.e., it has activity in a biological system and/or organism. For instance, a substance that, when introduced to an organism, has a biological effect on that organism, is considered to be biologically active or bioactive. In some embodiments, an agent to be delivered is an agent that inhibit, reduce or delay cell proliferation.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal and/or a clone.

Analogues or derivatives: As used herein, a derivative or an analogue refers to a compound can be formed from another compound. Typically, a derivative or an analogue of a compound is formed or can be formed by replacing at least one atom with another atom or a group of atoms. As used in connection with the present invention, a derivative of an analogue of a compound is a modified compound fat shares one or more chemical characteristics or features that are responsible for the activity of the compound. In some embodiments, a derivative or an analogue of a compound has a pharmacophore structure of the compound as defined using standard methods known in the art. In some embodiments, a derivative or an analogue of a compound has a pharmacophore structure of the compound with at least one side chain or ring linked to the pharmacophore that is present in the original compound (e.g., a functional group). In some embodiments, a derivative or an analogue of a compound has a pharmacophore structure of the compound with side chains or rings linked to the pharmacophore substantially similar to those present in the original compound. As used herein, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical linkage bonds (e.g., rotatable linkage bonds). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining the structures, or equivalent structures having a root mean square of deviation less than about 5.0 Å (e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining surface-accessible features (e.g., hydrogen bond donors and acceptors, charged/ionizable groups, and/or hydrophobic patches), or equivalent features leaving a root mean square of deviation less than about 5.0 Å (e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å).

Anti-neoplastic agent: As used herein, the term “anti-neoplastic agent” (also refer to as anti-proliferative agent) refers to an agent that inhibits and/or stops growth and/or proliferation of cells. An anti-neoplastic agent may display activity m vitro (e.g., when contacted with cells its culture), in vivo (e.g., when administered to a subject at risk of or suffering from hyperproliferation), or both. Exemplary anti-neoplastic agents include, but are not limited to, paclitaxel, enoxaprin, angiopeptin, carboplatin, vinorelbine, doxorubicin, gemcitabine, actinomycin-D, eisplatin, camptothecin, 5-fluorouracil, cyclophosphamide, 1-β-D-arabinofuranosylcytosine, or monoclonal antibodies capable of blocking smooth muscle cell proliferation, hirudin, and acetylsalicylic acid, amlodipine and doxazosin.

Combination therapy: The term “combination therapy”, as used, herein, refers to those situations in which two or more different pharmaceutical agents are administered in overlapping regimens so that the subject is simultaneously exposed to both agents.

Control: As used herein, the term “control” has its art-understood meaning of being a standard against which results are compared. Typically, controls are used to augment integrity is experiments by isolating variables in order to make a conclusion about such variables. In some embodiments, a control is a reaction or assay that is performed simultaneously with a test reaction or assay to provide a comparator.

Hyperproliferative condition: As used herein, the term “hyperproliferative condition” refers to undesirable cell growth. In some embodiments, hyperproliferative condition is associated with atherosclerosis, restenosis, proliferative vitreoretinopathy and psoriasis. The term is not intended to include cellular hyperproliferation associated with cancerous conditions. In some embodiments, undesirable cell growth refers to unregulated cell division associated with smooth muscle cells and/or fibroblasts. In some embodiments, undesirable cell growth is restenosis, which typically refers to the re-narrowing of opened artery after a surgical procedure such as stenting or PTCA procedure. Restenosis is typically due to a proliferative response of the intima, a layer of cells that line the lumen of the vessel, composed of connective tissue and smooth muscle cells (SMC).

Immunosuppressant agent: As used herein, the term “Immunosuppressant agent” refers to any agent that reduces, inhibits or delays an immuno-reaction such as an inflammatory reaction. Exemplary immunosuppressants include, but are not limited to, sirolimus (RAPAMYCIN), tacrolimus, everolimus, dexamethasone, zotarolimus, tacrolimus, everolimus, biolimus, pimecrolimus, supralimus, temsirolimus, TAFA 93, invamycin and neuroimmunophilins.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Polymer: As used herein, the term, “polymer” refers to any long-chain molecules containing small repeating units.

Prodrug: As used herein, the term “prodrug” refers to a pharmacological substance (drug) that is administered or delivered in an inactive (or significantly less active) form. Typically, once administered, the prodrug is metabolized in vivo into an active metabolite. The advantages of using prodrugs include better absorption, biocompatibility, distribution, metabolism, and excretion (ADME) optimization. Sometime, the use of a prodrug strategy increases the selectivity of the drug for its intended target.

Subject: As used herein, the term, “subject” or “patient” refers to any organism, to which systems, compositions or devices in accordance with the invention may be delivered or administered, e.g., for experimental, diagnostic, prophylactic, and/or therapeutic purposes. Typical subjects include animals (e.g., mammals such as mice, rats, rabbits, non-human primates, and humans; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained-release: As used herein, the term “sustained-release” refers to releasing (typically slowly) a drug over time. Typically, sustained-release formulations can keep steadier levels of the drug in the bloodstream. Typically, sustained-release coatings are formulated so that the bioactive agent is embedded in a matrix of polymers such that the dissolving agent has to find its way out through the holes in the matrix. In some embodiments, sustained-release coatings include several layers of polymers. In some embodiments, sustained-release coating matrix can physically swell up to form a gel, so that the drug has first to dissolve in matrix, then exit through the outer surface. As used herein, the terms of “sustained-release,” “extended-release,” “time-release” or “timed-release,” “controlled-release,” or “continuous-release” are used inter-changeably.

Therapeutically effective amount: As used herein, the terms “therapeutically effective amount” or “effective amount” of a therapeutic or bioactive agent refer to an amount that is sufficient when administered to a subject suffering from or susceptible to a disease, disorder, and/or condition, to treat, diagnose, prevent, and/or delay the onset of the symptom(s) of the disease, disorder, and/or condition. In some embodiments, an effective amount refers to the amount necessary or sufficient to inhibit the undesirable cell growth. The effective amount can vary depending on factors know to those of skill in the art, such as the type of cell growth, the mode and the regimen of administration, the size of the subject, the severity of the cell growth, etc.

Therapeutic agent: As used herein, the phrase “therapeutic agent” refers to any agent that, when administered to a subject, has a therapeutic effect and/or elicits a desired biological and/or pharmacological effect.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition (e.g., hyperproliferation such as restenosis). Treatment may be administered to a subject who does sot exhibit signs of a disease and/or exhibits only early signs of the disease for the purpose of decreasing the risk of developing pathology associated with the disease.

### DETAILED DESCRIPTION OF THE INVENTION

### Restenosis

Restenosis, e.g., In-Stent Restenosis (ISR), formation is a multi-factorial, sequential process. For example, it is generally believed that three stages are involved in the ISR process: 1) Thrombotic Phase (day 0-3 after stent implantation). This phase is the initial response of artery tissue to stent implantation characterized with rapid activation, adhesion, aggregation and deposition of platelets and neutrophils to form a thrombus in the injured site. 2) Recruitment Phase. This phase occurs between day 3 to 8 characterized with an intensive inflammation cell infiltration. In this phase, the inflammation cells winding leukocyte, monocytes, and macrophages were activated and infiltrated into the injured vessel wall. Subsequently, the recruited inflammation cells in the injured vessel wall provide the key stimulus for subsequent smooth muscle cell (SMC) proliferation and migration. In addition, the release and expression of adhesion cells, cytokines, chemokines, and growth factors by platelets, monocytes, and SMCs contribute to the further recruitment, infiltration at the site of injury, and further proliferation/migration of SMCs from media to neointima in the days after injuries. Anti-inflammation drugs (e.g., dexamethasone) and immunosuppressant drugs (e.g., sirolimus) are thought to delay or inhibit this phase. 3) Proliferate Phase. This phase last 1 to 3 months depending on the thickness of the residual thrombus and the rate of growth. At this stage, inflammation cells colonize the residual thrombus, forming a “cap” across the mural thrombus. The cells progressively proliferate, resorbing residual thrombus until all thrombus is gone and is replaced by the neointima tissue. These processes are induced by the early-phase events and also the exposure to circulatory mitogens (e.g., angiotensin II, plasmin). Vascular SMCs, otherwise in the quiescent phase of the cell cycle, are now triggered by early gene expression to undergo proliferation and migration with subsequent synthesis of extra cellular matrix and collagen, resulting in neointima formation. The process of neointimal growth, which consists of SMC, extracellular matrix, and macrophages recruited over a period of several weeks, is similar to the process of tumor tissue growth. This pathologic similarity between tumor cell growth and benign neointimal formation has led to the discovery of anti-tumor drugs as effective agents for the treatment of ISR.

### Sustained Drug Delivery Systems

A typical drug delivery system (also referred to as drug eluting system) for treating, preventing, inhibiting, or delaying the onset of retenosis include an implantable or insertable medical device (e.g., stent), coating or coating matrix, and bioactive agents. Implantable or insertable medical devices such as a stent provide a basic platform to deliver sufficient drug to the diseased arteries. Coating or coating matrix provides a reservoir for sustained delivery of bioactive agents. Typically, achieving compatibility between the implantable or insertable medical device, coating matrix, drugs and vessel wall is central for successful development of a drug delivery system.

### Implantable or Insertable Medical Devices

A typical platform for delivery of anti-restenosis drugs to an diseased arterial wall is an implantable or insertable medical device. A desirable drug-delivery platform typically has a larger surface area, minimal gaps between endothelial cells so as to minimize plaque prolapsed (displacement) in areas of large plaque burden, and minimal deformation (adaptation in shape or form) after implantation. Exemplary implantable or insertable medical devices suitable for the present invention include, but are not limited to, catheters, guide wires, balloons, filters, stents, stent grafts, vascular grafts, vascular patches or shunts.

In some embodiments, medical devices suitable for the invention are stents. Stents suitable for the present invention include any stent for medial purposes, which are known, to the skilled artisans. Exemplary stents include, but are not limited to, vascular stents such as self-expanding stents and balloon expandable stents. Examples of self-expanding stents useful in the present invention are illustrated in U.S. Pat Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

Suitable stents can be metal or non-metal stents. Exemplary biocompatible non-toxic metal stents include, but not limited to, stents made of stainless steel nitinol, tantalum, platinum, cobalt alloy, titanium, gold, a biocompatible metal alloy, iridium, silver, tungsten, or combinations thereof. Exemplary biocompatible non-metal stents include, but not limited to, stents made from carbon, carbon, fiber, cellulose acetate, cellulose nitrate, silicone, polyethylene teraphthalate, polyurethane, polyamide, polyester, polyorthoester, polyanhydride, polyether sulfone, polycarbonate, polypropylene, polyethylene, polytetrafluoroethylene, polylactic acid, polyglycolic acid a polyanhydride, polycaprolactone, polyhydroxybutyrate, or combinations thereof. Other polymers suitable for non-metal stents are shape-memory polymers, as described for example by Froix, U.S. Pat. No. 5,163,952, which is incorporated by reference herein. Stents formed of shape-memory polymers, which include methacylate-containing and acrylate-containing polymers, readily expand to assume a memory condition to expand and press against the lumen walls of a target vessel, as described by Phan, U.S. Pat. No. 5,603,722, which is incorporated by reference in its entirety.

Typically, implantable or insertable medical devices are adapted to serve as a structural support to carry a polymer based coating as described herein. For example, a polymer-based, drug containing fiber can be threaded through a metal stent aperture. The metal stent typically provides the mechanical support its the vessel after deployment for maintaining vessel patency, and the polymer thread provides a controlled release of bioactive agents. Another example is a drug-loaded polymer sheath encompassing a stent, as described in U.S. Pat. No. 5,383,928 (Scott, et al). Yet another example is a polymer stent which coexpand with a metal stent when placed in the target vessel, as described in U.S. Pat. No. 5,674,242 (Pham, et al).

The various embodiments of the present invention include implantable medical devices, such as stents, manufactured from polymers, more particularly, biodegradable polymers such as, without limitation, biodegradable polyesters, polyanhydrides, or poly(ether-esters). The polymer may be a biostable polymer, a biodegradable polymer, or a blend of a biostable polymer and a biodegradable polymer. As noted above, processing of a polymer, such as, without limitation, poly(L-lactide) (PLLA), results in the polymer being exposed to elevated temperatures, moisture, viscous shear, and other potential sources of degradation, such as metals and metal catalysts. Certain embodiments of the present invention involve the addition of one or more therapeutic agent to the polymer before and/or during the manufacturing process.

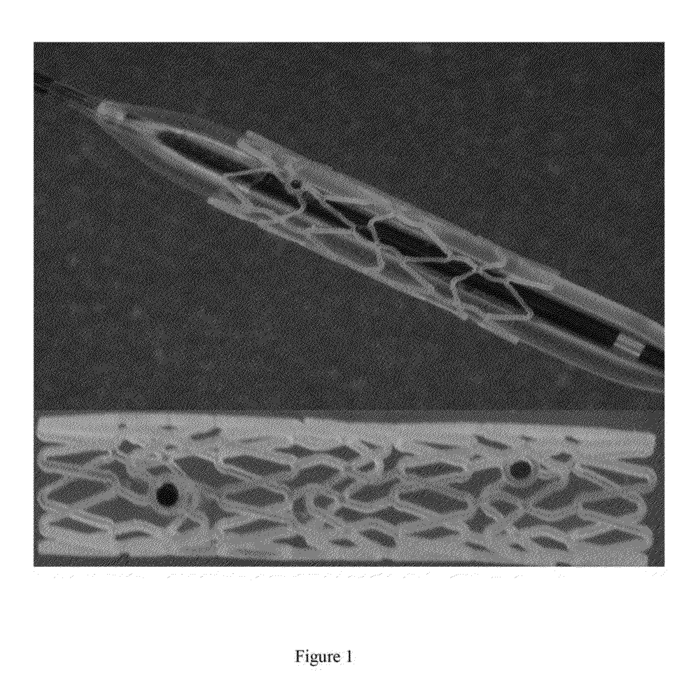

A stent may include a pattern or network of interconnecting structural elements or struts. FIG. 1 depicts on example of a three-dimensional view of a stent, The stent may have a pattern that includes a number of interconnecting elements or struts 1. The embodiments disclosed herein are not limited to stents or to the stent pattern illustrated in FIG. 1.

Although the discussion that follows focuses on a stent its an example of an Implantable medical device, the embodiments described herein are easily applicable to other implantable medical devices, including, but not limited to self-expandable stents, balloon-expandable stents, stent-grafts, and grafts. The embodiments descried herein are easily applicable to patterns other than that depicted in FIG. 1. The structural pattern of the device can be of virtually any design. The variations in the structure of patterns are virtually unlimited.

### Polymers

Polymers suitable for the drug—incorporation of the present invention include any polymers that are biologically inert and not induce further inflammation (e.g., biocompatible and avoids irritation to body tissue). In some embodiments, suitable polymers are non-biodegradable. Exemplary non-biodegradable polymers include, but are not limited to, poly-n-butyl methacrylate (PBMA), polyethylene-co-vinyl acetate (PEVA), poly(styrene-b-isobutylene-b-styrene (SIBS), and combinations or analogues thereof.

Other non-biodegradable polymers that are suitable for use in this invention include polymers such as polyurethane, silicones, polyesters, polyolefins, polyamides, polycaprolactam, polyimide, polyvinyl chloride, polyvinyl methyl ether, polyvinyl alcohol, acrylic polymers and copolymers, polyacrylonitrile, polystyrene copolymers of vinyl monomers with olefins (such as styrene acrylonitrile copolymers, ethylene methyl methacrylate copolymers, ethylene vinyl acetate), polyethers, rayons, cellulosics (such as cellulose acetate, cellulose nitrate, cellulose propionate, etc.), parylene and derivatives thereof; and mixtures and copolymers of the foregoing.

In some embodiments, a suitable biodegradable polymer is a polyester. Exemplary polyester polymers suitable for the invention include, but are not limited to, poly(L-lactide), poly (D,L-lactide), poly(L-lactide-co-D,L-lactide), poly(L-lactide-co-glycolide), poly(D,L-lactide-co-glycolide), poly(L-lactide-co-caprolactone), poly(glycolide-co-caprolactone), poly(D,L-lactide-co-caprolactone) and blends of the aforementioned. PLA and PGA are desirable for medical applications because they have lactic acid and glycolic acid as their degradation products, respectively. These natural metabolites are ultimately converted to water and carbon dioxide through the action of enzymes in the tricarboxylic acid cycle and are excreted via the respiratory system. In addition, PGA is also partly broken down through the activity of esterases and excreted in the urine. Along with its superior hydrophobicity, PLA is more resistant to hydrolytic attack than PGA, making an increase of the PLA:PGA ratio in a PLGA copolymer result in delayed degradability.

Thus, although the invention can be practiced by using a single type of polymer, it is desirable to use various combinations of polymers. The appropriate mixture of polymers can be coordinated with biologically active materials of interest to produce desired effects in accordance with the invention.

In some embodiments, polymers suitable for the invention include calcium phosphates. In some embodiments, calcium phosphates are used in combination with biodegradable polymers. Without wishing to be bound to a particular theory, it is believed that combining calcium phosphate material with biodegradable polymers may buffer the acidic materials released by biodegradation, and therefore provide the polymer that will induce less inflammation. In some embodiments, the ratio of the polyester polymer and the calcium phosphate ranges from about 99:1 to 1:99 (e.g., 10:90. 20:80, 30:70, 40:60, 50:50, 60:40, 70:30, 80:20, 90:10).

Exemplary calcium phosphates that may be used in the current invention include, but not limited to, amorphous calcium phosphate (ACP), dicalcium phosphate (DCP), tricalcium phosphate (TCP), pentacalcium hydroxyl Apatite (HAp), tetracalcium phosphate monoxide (TTCP) and combinations or analogues thereof.

For example, ACP is an important intermediate product for in vitro and in vivo apatite formation with high solubility and better biodegradability. It was mainly used in the form of particles or powders, as an inorganic component incorporated into biopolymers, to adjust the mechanical properties, biodegradability, and bioactivity of the resulting composites. Based on the similarity of ACP to the inorganic component of the bone, ACP is particular useful as a bioactive additive in medical devices to improve remineralization. Based on its solubility, coatings containing ACP may release ions into aqueous media, forming a favorable super saturation level of Ca²⁺ and PO₄³⁻ ions for the formation of apatite. The ion release may neutralize the acidity resulted from polymer biodegradation, retarding bioresorptive rate and eliminating inflammation occurrence.

### Therapeutic Agents

The current invention provides encapsulating at least an anti-neoplastic agent and/or an immunosuppressant agent in to a polymer. In some embodiments, an anti-neoplastic agent suitable for the invention is paclitaxel, or a prodrug or analog thereof. In some embodiments, anti-neoplastic agents suitable for the invention is selected from, carboplatin, vinorelbine, doxorubicin, gemcitabine, actinomycin-D, cisplatin, camptothecin, 5-fluorouracil cyclophosphamide, 1-β-D-arabinofuranosylcytosine, or a combination or analogs thereof. In some embodiments, an immunosuppressant agent suitable for the invention is sirolimus, or a prodrug or analog thereof. In some embodiments, immunosuppressant agents suitable for the invention is selected from zotarolimus, tacrolimus, everolimus, biolimus, pimecrolimus, supralimus, temsirolimus, TAFA 93, invamycin or neuroimmunophilins, or a combination or analogs thereof.

Paclitaxel, an extract from the bark of the Pacific yew tree Taxus brevifolia, has a melting point of 220 degree C. The anti-proliferative activity of paclitaxel is a result of concentration-dependent and reversible binding to microtubules, specifically to the β-subunit of tubulin at the N-terminal domain. This binding promotes polymerization of tubulin to form stable microtubules by reducing the critical concentration of tubulin required for polymerization and preventing depolymerization of the microtubules; the structure of the microtubules is stabilized by the formation of bundles and multiple asters.

Paclitaxel produces distinct dose-dependent effects within the cell: at low doses it causes G₁ arrest during interphase by inducing p53 and p21 tumor suppression genes, resulting in cytostasis. At high doses, the drug is thought to affect the G₂-M phase of the cell cycle. Since the microtubules must be disassembled for transition from the G2 to the M phase to take place, and paclitaxel stabilizes the microtubule structure, mitotic arrest occurs in the presence of paclitaxel. Alternatively, high doses may affect the M-G₁ phase causing post-mitotic arrest and possibly apoptosis. In addition to these actions, activation of some protein kinases and serine protein phosphorylation are associated with depolymerization of microtubules, and are therefore inhibited by paclitaxel. Thus, any paclitaxel analogs that retain or improve the cell cycle inhibitory function of paclitaxel as described herein can be used in accordance with the invention.

Sirolimus (rapamycin), a natural macrolide antibiotic with potent immunosuppressant properties, has a melting point of 180 degree C. Sirolimus was first approved by the FDA in 1999 for use as an anti-rejection agent following organ transplantation. Its use in intracoronary stenting was based on the premise that the anti-proliferative properties of the drug would inhibit the neointimal hyperplasia (NIH) associated with restenosis following stent implantation. An important mechanism of Sirolimus action k entry into target cells and binding to the cytosolic immunophilin FK-binding protein-12 (FKBP-12) to form a Siroliomus:FKBP-12 complex that interrupts signal transduction, selectively interfering with protein synthesis. After binding with FK-binding protein-12 (FKBP-12), Sirolimus inhibits the activity of the mammalian target of Rapamycin (mTOR) and eventually the activity of the cyclin-dependent kinase (cdk)/cyclin complexes, as well as the phosphorylation of retinoblastoma protein, thereby preventing advancement of the cell cycle from G1 to S phase. Thus, any Sirolimus analogs that retain or improve the cell cycle inhibitory function of Sirolimus as described herein can be used in accordance with the invention.

In preferred embodiments, the present invention provides drug-containing polymeric compositions containing a combination of an anti-neoplastic agent (such as paclitaxel or its prodrug or analogs) and an immunosuppressant agent (such as sirolimus or its prodrug or analogs).

Several combination therapies have been investigated previously in the treatment of in-stent restenosis. However, all those investigations involved the combination of anti-plastic (Paclitaxol) or immunosuppressant drug (Sirolimus) with anti-thrombotic agents such as Glycoprotein IIB/IIIA inhibitor or heparin) ( Leon M B and Bakhai Ameet, “Drug releasing stent and glycoprotein IIb/IIA inhibitor: combination therapy for the future,” Am Heart J 2003; 146: S13-7) or nitric oxide (Lin-Chiaen, and Delano Yang et al. “Combination of paclitaxel and nitric oxide as a novel treatment for the reduction of restenosis,” J. Med. Chem. 2004; 47: 2276-2282). The purpose of adding anti-thrombotic drugs to coated stem is to prevent thrombosis. However, the efficacies of these combinations as inhibition of neointimal hyperplasia alter stent implantation are limited. The one possible reason for the limited effects of these combinations is the physiochemical incompatibility among combined drugs. Local drugs that are retained within the blood vessel are more effective than those are not. Both heparin and nitric oxide compounds are so soluble and diffusible that they simply cannot stay in the artery for more than a few minutes after release. US patent application to Hsu Li-Chien (US-2004/0037886: Drug Eluting Stent for Medical Implant) had disclosed a modified coating system to increase the compatibility among combined drugs (hydrophilic and hydrophobic drugs). However, as discussed below, the combination used in the modified coating system in Hsu's patent application is completely different from the combination therapies contemplated in the present application.

Drug-Containing polymeric composition of the present invention is developed to harness synergistic effects between an anti-neoplastic agent and an immunosuppressant agent. For example, contrary to the above-described hydropholic and hydrophobic drug combinations, both sirolimus and paclitaxel are hydrophobic, and retained well in blood vessel wall for up to three days through specifically binding to their individual binding proteins (Levin, A. D. et al., “Edelman Specific binding to intracellular proteins determines arterial transport properties for rapamycin and paclitaxel,” PNAS 2004; 101 (25): 9463-67) after releasing from stent. Therefore, it is contemplated that a combination of these two drugs in a coating according to the invention may work synergistically to inhibit restenosis including neointimal hyperplasia. Medical devices encapsulated with a combination of bioactive agents would require fewer doses of each agent to achieve the same or even greater anti-restenosis effects with less side-effects compared to otherwise identical medical devices coated with individual agent alone. The detail compositions combining anti-neoplastic agents and an immunosuppressant agents such as sirolimus and paclitaxel or prodrugs or analogs thereof, are described in the U.S. application Ser. No. 11/144,917. Additional coating formulations containing anti-neoplastic agents and an immunosuppressant agents such as sirolimus and paclitaxel or prodrugs or analogs thereof, and biodegradable polymers are described in U.S. patent application Ser. No. 11/843,528.

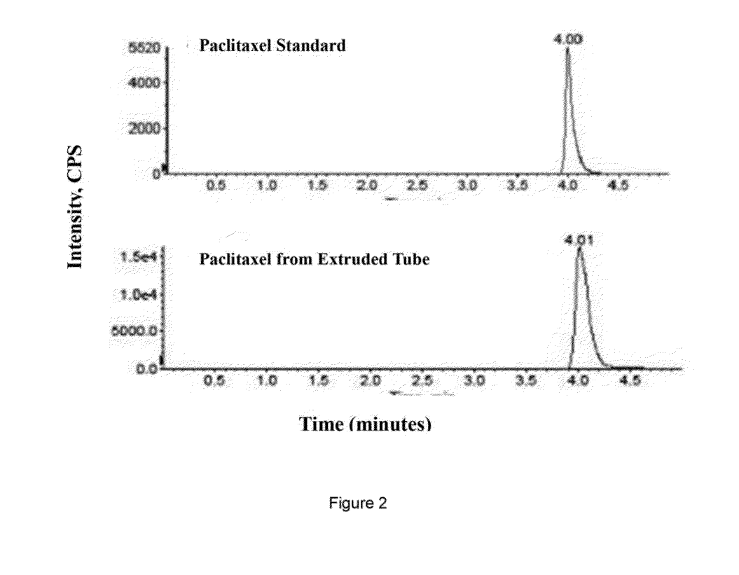

The present invention, further demonstrated that both sirolimus and paclitaxel can be incorporated into polymeric stent strut through extrusion process and released in a controlled manner. As shown FIG. 2, paclitaxel in a invented drug impregnated biodegradable stent survived the elevated extrusion temperature and are stable inside the stent strut. Therefore, the present invention provides new and powerful drug-eluting system for treatment of restenosis and an extrusion process for making the same.

Bioactive agents suitable for the invention may also include anti-thrombogenic agents such as heparin, heparin derivatives, urokinase, and PPack (dextrophenylalanine proline arginine chloromethylketone); anti-inflammatory agents such as glucocorticoids, betamethasone, dexamethasone, prednisolone, corticosterone, budesonide, estrogen, sulfasalazine, and mesalamine; other antineoplastic/antiproliferative/anti-miotic agents such as 5-fluoroaracil, eisplatin, vinblastine, vineristine, epothilones, methotrexate, azathioprine, halofuginone, adriamycin, actinomycin and mutamycin; endostatin, angiostatin and thymidine kinase inhibitors, and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promoters such as growth factors. Vascular Endothelial Growth Factors (FEGF, all types including VEGF-2), growth factor receptors, transcriptional activators, and translational promoters; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules including a growth factor and a cytotoxin, bifunctional molecules including an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

In addition, bioactive agents suitable for the present invention include nitric oxide adducts, which prevent and/or treat adverse effects associated with use of a medical device in a patient, such as restenosis and damaged blood vessel surface. Typical nitric oxide adducts include, but are not limited to, nitroglycerin, sodium nitroprusside, S-nitroso-proteins, S-nitroso-thiols, long carbon-chain lipophilic S-nitrosothiols, S-nitrosodithiols, iron-nitrosyl compounds, thionitrates, thionitrites, sydnonimines, furoxans, organic nitrates, and nitrosated amino acids, preferably mono-or poly-nitrosylated proteins, particularly polynitrosated albumin or polymers or aggregates thereof. The albumin is preferably human or bovine, including humanized bovine serum-albumin. Such nitric oxide adducts are disclosed in U.S. Pat. No. 6,087,479 to Stamler et al. which is incorporated herein by reference.

Bioactive agents may be encapsulated in micro or nano-capsules by the known methods.

Bioactive agents can be used with (a) biologically non-active material(s) including a carrier or an excipient, such as sucrose acetate isobutyrate (SABER™ commercially available from SBS) ethanol n-methyl pymolidone, dimethyl sulfoxide, benzyl benxoate, benzyl acetate, albumine, carbohydrate, and polysaccharide. Also, nanoparticles of the biologically active materials and non-active materials are useful for the coating formulation of the present invention.

Bioactive agents including anti-neoplastic agents and immunosuppressant agents may be present in one single layer. Alternatively, individual agents (such as anti-neoplastic agents and immunosuppressant agents) may be present in separate layers. In some embodiments, a drug-free polymer layer (also referred to as cap layer) can be coated over a layer or layers containing an anti-neoplastic agent and/or immunosuppressant agent to act as a diffusion barrier.

### Crystallized Polymeric and Drug Nanoparticle Preparation

The at least one biocompatible polymer of the present invention may form polymeric particles with the at least one therapeutic agent encapsulated therein. The polymeric particles may have any suitable sixes (e.g., from about 1 nm to about 1 mm in average diameter) and shapes (e.g., sphere, ellipsoid, etc.). Preferably, but not necessarily, the at least one biocompatible polymer of the present invention forms nano- and/or micro-particles that are suitable for injection. The term “nano-particles” or “micro-particles” is used throughout the present invention to denote carrier structures that are biocompatible and have sufficient resistance to chemical and/or physical destruction by the environment of use such that a sufficient amount of the nano-particles and/or micro-particles remain substantially intact after injection into a target site in the arterial wall. Typically, the nano-particles of the present invention have sizes ranging from about 1 nm to about 1000 nm, with sixes from about 100 nm to about 500 nm being more preferred. The micro-particles of the present invention, have sizes ranging from about 1 .mu.m to about 1000 .mu.m, with sizes from about 10 .mu.m to about 200 .mu.m being more preferred. The pharmacologically active agent as described hereinabove is loaded within and/or on the surfaces of the nano-particles and/or micro-particles.

In a particularly preferred embodiment of the present invention, the at least one therapeutic agent are first formed into crystalline particles of desired sizes, and are then encapsulated into the at least one biocompatible polymer through extrusion or injection molding process. Preferably, but not necessarily, the crystalline particles of the therapeutic agent have an average particle size ranging from about 50 nm to about 50 .mu.m, and more preferably from about 100 nm to about 200 nm.

In order to retain the physical properties of the drug-containing devices (polymer film or coating integrity, etc), it may be necessary to reduce the particle size of the therapeutic agents. Smaller drug particle size will also provide different drug formulation and processing options, without affecting the processing efficiency. Crystalline drug particles with the desired particle sizes can be readily formed by several different processes, as described hereinafter.

Nanotechnology provides new and enhanced particle formulation processes and offers a wide range of options for achieving drug particles in the micro- and nano-size range. Some of the new developments in nanotechnology have successfully achieved particle engineering by using molecular scaffolds like dendrimers (polyvalent molecules) and fullerenes (i.e., C-60 “bucky balls”). The small-size drug particles that can be formed by using nanotechnology are particularly useful for formulating poorly soluble drugs, since the reduced drug particle sizes significantly improve the bioavailability of such drugs, by providing higher surface area and accelerating dissolution and absorption of such drugs by the body.

Further, conventional techniques, such as milling (either dry or wet), supercritical extraction, spray drying, precipitation, and recrystallization, can also be used to prepare micro- and nano-size drug particles.

Milling is a well-established micronization technique for obtaining desired, micro- and nano-size drug particles (either dry or suspended in liquid) with well controlled size distribution.

Dry milling can be used to obtain particle size below about 50 microns. Various dry milling methods, such as jet milling, high-speed mixer milling, planetary milling, fluid energy jet milling, and ball milling, can be used to grind drug particles to about 1 micron. Milling is a relatively less expensive, faster, and easily scalable method, in comparison with other methods. Micronization occurs by particle collision (e.g., particle-particle or collisions among the particles and the grinding media like balls, pins, or heads) in various vessel configurations that may be stationary or shaken, rolled, or spun. These processes may involve compressed steam, compressed nitrogen, or compressed air. Process variables include air pressure used for grinding, time in the grinding stone and the feed rate.

Wet milling can be used to form solid drug particles below 1 micron to 80-150 nm with well defined size distribution. Bead milling uses rotating agitator disks to move microsized grinding beads (50 microns to 3.0 mm) in an enclosed grinding chamber to produce particles as small as 0.1 micron. Another wet-milling system (NanoCrystal™. System developed by Elan Drug Delivery) used for poorly water-soluble drugs generates particles sized in the 100-200 nm range.

Supercritical fluids (SCF) can also be used to form small-size drug particles, by extracting solvents from dissolved drugs while drug-containing droplets are sprayed out of a nozzle. The anti-solvent used for extraction is typically supercritical carbon dioxide, and the solvent(s) is typically water, ethanol, methanol, or isopropyl alcohol. No solvent is used if the drug is readily soluble in compressed carbon dioxide. In this event, the drug-containing supercritical carbon dioxide simply is sprayed into a depressurized vessel. The particle-formation rate can be controlled by changing the pressure, temperature, and spray rate. The particle size is determined mainly by the size of the droplet and the choice of the SCF. Dissolving the same drug into two different solvents may result in two different particle sizes. Particle sizes ranges typically in the range of about 100 nm. Crystalline morphology of the drug particles is retained by careful control over the small period of time when a drug comes out of solution and forms the particles.

Spray-drying technology is similar to the SCF approach, except that instead of using a SCF to remove the solvent(s), the solvent(s) is removed by a controlled drying process. A drug and excipient formulation is dissolved in a solvent or a mixture of two or more solvents. The solution is then sprayed through a nozzle, forming very fine droplets, which are passed down a drying chamber at either elevated or reduced temperatures. A drying gas, such as nitrogen, causes the solvent(s) to precipitate from the droplets, resulting in dry drug particles. One particularly preferred spray-drying method uses a multichamber spray dryer to produce porous microspheres. The chambers are arranged in series, so that the particles can be dried sequentially at different temperatures. The crystallinity of the drug particles is retained by controlling the chamber temperatures and the drying conditions.

Spray drying can generate particles with mean size ranges from 700 nm to 2-3 microns. Spray drying can be used with either water-soluble or insoluble drugs.

Precipitation is another technique that can be used to form small-sized drug particles from solution. One precipitation technique specifically uses low-frequency sonication to speed up the precipitation process, by producing a homogenous shear field inside the vessel. A drug-containing solution is introduced into a vessel sitting on a magnetic plate oscillating at frequencies typically around 60 Hz. The frequency facilitates the precipitation of the drug particles, which can then be dried or filtered. Precipitation can also be achieved by pH shift, by using a different solvent, or by changing the temperature. The oscillation frequency, the volume, and the manner in which the precipitation is achieved can be readily adjusted to form drug particles of the desired particle sizes. The particle size achieved by precipitation is typically in the range of 400 to 600 nm.

If the particle sizes of the crystalline drug particles as provided are already suitable for forming a polymeric composition that can be subsequently used to form a drug-eluting implantable medical device, then such crystalline drug particles can be directly used for forming the polymeric composition. However, if the particle sizes of the crystalline drug particles as provided are too large, the above-described methods can be readily used, either separately or in combination, to reduce the particles size down to a desired size range.

The drug-containing polymeric composition of the present invention can be formed by various methods that effectively encapsulate the small-size crystalline drug particles, as described hereinabove, into at least one biocompatible polymer as described hereinabove, provided that during and after the processing steps of such methods, at least a portion of the crystalline particles remain crystalline. Preferably more than 50%, more preferably more than 75%, and most preferably more than 90% of the crystalline particles remain crystalline during and after use processing steps of such methods.

### Fabrication of Drug Eluting Stent

A stent such as stent 1 may be fabricated from a polymeric tube or a sheet by rolling and bonding the sheet to form the tube. A tube or sheet can be formed by extrusion or injection molding. A stent pattern, such as the one pictured in FIG. 1, can be formed in a tube or sheet with a technique such as laser cutting, machining or chemical etching. The stent can then be crimped on to a balloon or catheter for delivery into a bodily lumen.

The elevated temperatures, exposure to shear, exposure to moisture and exposure to radiation that is encountered in polymer processing may lead to degradation of both the polymer and the drugs. Such degradation may lead to a decrease b polymer molecular weight, drug stability. In addition, polymer and drug degradation can result in formation of oligomers, cyclic dimers, and monomers, with or without a significant decrease in molecular weight, which can alter the polymer and drug properties and degradation behavior.

Some of the process operations involved in fabricating a drug-delivery stent may include:

(1) forming a drug-containing polymeric tube using extrusion;

(2) radially deforming the formed drug-containing tube by application of heat and/or pressure;

(3) forming a stent from the deformed tube by cutting a stent pattern in the deformed tube;

(5) crimping the stent on a support element, such as a balloon on a delivery catheter;

(6) packaging the crimped stent/catheter assembly; and

(7) sterilizing the stent assembly.

### Extrusion/Injection Molding

The initial step in the manufacture of a drug-delivery stent is to obtain a drug-containing polymer tube or sheet. The polymer tube or sheet may be formed using various types of forming methods, including, but not limited to, extrusion or injection molding. A polymer sheet may be rolled and bonded to form a polymer tube. Representative examples of extruders include, but are not limited to, single screw extruders, intermeshing co-rotating and counter-rotating twin-screw extruders and other multiple screw masticating extruders.

Both extrusion and injection molding expose the drug-polymer composition to elevated temperatures and shear. In extrusion, a drug-polymer composition melt is conveyed through an extruder and forced through a die as a film in the shape of a tube. Depending upon the type of extrusion and the molecular weight of the polymer, the polymer may be close to, at, or above its melting point. Specifically, the melt viscosity is desirably in a particular range to facilitate the extrusion process. In general, as the molecular weight increases, higher processing temperatures may be needed to achieve a melt viscosity that allows for processing. For example, for a biodegradable polyester such as poly(L-lactide), the temperature range may be in the range of about 180.degree. C. to 220.degree. C. for a melt extrusion operation. The residence time in the extruder may be about 5 minutes to about 30 minutes. These high temperatures, combined with the shear, moisture, residual catalyst, and other metals to which the drug-polymer matrix is exposed during extrusion, may lead to polymer degradation and drug decomposition.

The extrusion process can be used in the present invention to form drug-containing polymeric tube of desired drug release profile (e.g., either an immediate release profile or a controlled release profile), depending on the polymer used. Further, each polymeric can contain two or more active drugs. Alternatively, two or more active ingredients that may potentially interact with one another in an undesired manner (i.e., incompatible) can be encapsulated into separate layer by multiple extrusion technology.

Specifically, a biocompatible polymer, which has a lower melting temperature than the therapeutic agent to be encapsulated, is melted, and the melted polymer is then mixed with the crystalline particle of the therapeutic agent to form a molten mixture. Since the therapeutic agent has a higher melting temperature than the polymer, the crystallinity of the therapeutic particles is not affected by mixing with the melted polymer. Subsequently, the molten mixture is extruded into a tube, and then cooled to below the melting temperature of the biocompatible polymer, thereby forming a solidified tubular structure that comprises a substantially continuous polymeric matrix with the crystalline particles of the therapeutic agent encapsulated therein. The solidified tube structure can be treated by various techniques, such as, annealing, deforming, and laser cutting etc.

Any biocompatible polymer or polymer blends that has a melting temperature lower than that of the therapeutic agent can be used in the above-described melt compounding process. For example, poly(lactide-co-glycolide), which has a processing temperature of about 150.degree. C., can be used for melt compounding with both rapamycin (i.e., sirolimus), which has a melting temperature of about 180.degree. C. and paclitaxel which has a melting temperature of 220 degree C. while PLLA, which has a processing temperature of about 180 to 190.degree. C., can be used for melt compounding with paclitaxel only. For another example, Poly(glycolide-caprolactone) copolymer (65/35), which has a processing temperature of about 120.degree. C., can be used for melt compounding with cladribine, which has a melting temperature of about 220.degree. C. Poly(caprolactone-dioxanone) copolymer (95/5), which has a processing temperature of about 80 to 100.degree. C., can be used for melt compounding with sabeluzole, which has a melting temperature of about 110.degree. C.

Therefore, in one aspect of the present invention is to provide methods to maintain drug-containing tube, or at least a portion thereof, in the more stable crystalline phase. Preferably, but not necessarily, the drug-containing polymeric tube of the present invention contain little or no amorphous therapeutic agents, i.e., a major portion (i.e., >50%) of the therapeutic agents contained in such compositions are in the stable crystalline phase. For example, the drug-containing polymeric tube of the present invention each comprises at least one therapeutic agent encapsulated in at least one biocompatible polymer, while more than 75% of the therapeutic agent in the composition is crystalline. More preferably, more than 90% or more than 95% of the therapeutic agent in the composition is crystalline. Most preferably, the composition is essentially free of amorphous therapeutic agent.

### Polymeric and Drug Molecular Orientation

Generally, application of strain radically and axially can induce both the polymer and drug molecular orientation along the direction of strain which can increase the strength and modulus along the direction of strain.

A technique for the radial axial deformation of a tube is blow molding. The polymeric tube is placed in a mold, and applied strain axially. The tube is deformed in the both radial and axial direction by application of a pressure from a air. The pressure expands the tube such that it contacts the walls of the mold, the strain strength the tube axially. The mold may act to limit the radial deformation of the polymeric tube to a particular diameter, the inside diameter of mold. And the expansion was controlled by the weight applied to the tube.

During the blow molding, the polymer tube may be heated by a heated gas or fluid or water, or the mold may be heated, thus heating the polymer tube within. After the tube has been blow molded to a particular diameter, the tube can be maintained under the elevated pressure and temperature for a period of time. The period of time may be between about one minute and about one hour, or more narrowly, between about two minutes and about ten minutes. This is referred to as “heat setting.”

As polymer chains have greater mobility above T.sub.g, maintaining the polymer tube in a deformed state at a temperature above the T.sub.g, that s heat setting the tube, allows the chains to rearrange closer to a thermodynamically equilibrium condition. Also, for polymers that are capable of crystallization, crystallization occurs at temperatures between the glass transition temperature and the melting temperature.

Thus, during radial and axial expansion the tube may be at a temperature between the glass transition temperature and the melting temperature. After expansion, the tube may remain in the mold for a period of time at the elevated temperature of expansion. As an example, the polymer may be exposed to a temperature of about 80.degree. C. to 160.degree. C. for the duration of processing, about 3-15 minutes, and optionally heat set afterwards.

### Stent Cutting

Once the polymeric tube has been formed, and optionally radially expanded, a stent pattern is cut into the tube. The stent pattern may be formed by any number of methods including chemical etching, machining, and laser cutting. Laser cutting generally results in a heat affected zone (HAZ). A HAZ refers to a portion of a target substrate that is not removed, but is still exposed to energy from the laser beam, either directly or indirectly. Direct exposure may be due to exposure to the substrate from a section of the beam with an intensity that is not great enough to remove substrate material through either a thermal or nonthermal mechanism. A substrate can also be exposed to energy indirectly due to thermal conduction and scattered radiation. The exposure to increased temperature in a HAZ sway lead to polymer degradation.

In some embodiments, the extent of a HAZ may be decreased by the use of an ultrashort-pulse laser. This is primarily due to the increase in laser intensity associated with the ultrashort pulse. The increased intensity results in greater local absorption. “Ultrashort-pulse lasers” refer to lasers having pulses with durations shorter than about a picosecond (=10.su.−12) and includes both picosecond and femtosecond (=10.sup.−15) lasers. Other embodiments include laser machining a stent pattern with a conventional continuous wave or long-pulse laser (nanosecond (10.sup.−9) laser) which has significantly longer pulses than ultrashort pulse lasers. There is a larger HAZ for a continuous or long-pulse laser as compared to an ultrashort pulse laser, and therefore the extent of polymer degradation is higher.

Further embodiments can include fabricating a stent delivery device by crimping the stent on a support element, such as a catheter balloon, such that the temperature of the stent during crimping is above an ambient temperature. Heating a stent during crimping can reduce or eliminate radially outward recoiling of a crimped stent which can result in an unacceptable profile for delivery. Crimping may also occur at an ambient temperature. Thus, crimping may occur at a temperature ranging from 30.degree. C. to 60.degree. C. for a duration ranging from about 60 seconds to about 5 minutes.

Once the stent has been crimped onto a support element, such as without limitation, a catheter balloon, the stent delivery device is packaged and then sterilized. Ethylene oxide sterilization, or irradiation, either gamma irradiation or electron beam irradiation (e-beam irradiation), are typically used for terminal sterilization of medical devices. For ethylene oxide sterilization, the medical device is exposed to liquid or gas ethylene oxide that sterilizes through an alkalization reaction that prevents organisms from reproducing. Ethylene oxide penetrates the device, and then the device is aerated to assure very low residual levels of ethylene oxide because it is highly toxic. Thus, the ethylene oxide sterilization is often performed at elevated temperatures to speed up the process. Moisture is also added as it increases the effectiveness of ethylene oxide in eliminating microorganisms. Polymer degradation may occur due to the ethylene oxide itself interacting clinically with the polymer, as well as result from higher temperatures and the plasticization of the polymer resulting from absorption of ethylene oxide. More importantly, polymer degradation can occur from the combination of heat and moisture.

Alternatively, irradiation may be used for terminal sterilization. It is known that radiation can alter the properties of the polymers being treated by the radiation. High-energy radiation tends to produce ionization and excitation in polymer molecules. These energy-rich species undergo dissociation, subtraction, and addition reactions in a sequence leading to chemical stability. The degradation process can occur during, immediately after, or even days, weeks, or months after irradiation which often results in physical and chemical cross-linking or chain scission. Resultant physical changes can include embrittlement, discoloration, odor generation, stiffening, and softening, among others.

In particular, the deterioration of the performance of polymers due to e-beam radiation sterilization has been associated with free radical formation during radiation exposure and by reaction with other parts of the polymer chains. The reaction is dependent on e-beam dose, temperature, and atmosphere present. Additionally, exposure to radiation, such as e-beam, can cause a rise in temperature of an irradiated polymer sample. The rise in temperature is dependent on the level of exposure, in particular, the effect of radiation on mechanical properties may become more pronounced as the temperature approaches and surpasses the glass transition temperature, T.sub.g. The deterioration of mechanical properties may result from the effect of the temperature on polymer morphology, but also from increased degradation resulting in a decrease in molecular weight. As noted above, degradation may increase above the glass transition temperature due to the greater polymer chain mobility.

Thus, in some embodiments sterilization by irradiation, such as with an electron beam, may be performed at a temperature below ambient temperature. As an example, without limitation, sterilization may occur at a temperature at the range of about −30.degree. C. to about 0.degree. C. Alternatively, the stent may be cooled to a temperature in the range of about −30.degree. C, to about 0.degree. C., and then sterilized by e-beam irradiation. The sterilization, may occur in multiple passes through the electron beam. In other embodiments, sterilization by irradiation, such as with an electron beam, may occur at ambient temperature.

As outlined above, the manufacturing process results in the polymer and drug's exposure to high temperatures and other potential sources of degradation, such as without limitation, irradiation, moisture, and exposure to solvents. In addition, residual catalysts in the polymer raw material, and other metals, such as from processing equipment, may catalyze degradation reactions. The polymer and drug are also exposed to shear stress, particularly during extrusion. Thus, there are a number of sources of potential polymer and drug degradation.

Polymer molecular weight may significantly decrease during the processing operations used in the manufacture of a stent. A non-limiting example is the use of a PLLA polymer to manufacture a stent. The stent manufacturing process involves extruding a polymer tube, radially expanding the polymer tube, laser cutting a stent pattern into the tube to form a stent, crimping the stent onto a balloon catheter, and sterilizing the crimped stent. The entire process results to a decrease of the weight average molecular weight from about 550 kg/mol to about 190 kg/mol. Extrusion of the polymer tube results in a decreases to about 380 Kg/mol from the initial 550 kg/mol. The molecular weight is further decreased to about 280 kg/mol after radial expansion and laser cutting. After sterilization by electron beam irradiation (25 KGy), the molecular weight (weight average) is about 190 kg/mol.

In general the decomposition of a polymer, for example a biodegradable polyester such as, without limitation, PLLA, is due to exposure to heat, light, radiation, moisture, or other factors. As a result, a series of byproducts such as lactide monomers, cyclic oligomers and shorter polymer chains appear once the formed free radicals attack the polymer chain. In addition, decomposition may be catalyzed by the presence of oxygen, water, or residual metal such as from a catalyst. More specifically the polyester poly(L-lactide) is subject to thermal degradation at elevated temperatures, with significant degradation (measured as weight loss) occurring at about 150.degree. C. and higher temperatures. The polymer is subject to random chain scission. To explain the presence of lactide at higher temperatures, some have postulated the existence of an equilibrium between the lactide monomer and the polymer chain. In addition to lactide, the degradation products also include aldehydes, and other cyclic oligomers. Although the degradation mechanisms of PLLA are not fully understood, a free radical chain process can be involved in the degradation. Other mechanisms include depolymerization due to attack by the hydroxyl groups at the chain ends, ester hydrolysis occurring anywhere on the polymer due to water, and thermally driven depolymerization occurring anywhere along the polymer chain. In the cases of depolymerization occurring by backbiting from the terminal hydroxyl groups or thermally driven a long the polymer backbone, these process may be especially accelerated by the presence of polymerization catalysts, metal ions, and Lewis acid species.

In some embodiments, the fabrication, of the implantable medical device may include at least one melt processing operation, while others may include at least two operations where the processing temperature is above the glass transition temperature of the polymer. In some embodiments, the fabrication of the implantable medical device may include at least one melt processing operation and at least one additional operation where the processing temperature is above the glass transition temperature of the polymer. The various processing operators may occur at a temperature of at least 160.degree. C., at least 180.degree. C., at least 200.degree. C., or at least 210.degree. C.

In some embodiments, the fabrication of the implantable medical device may include any of the processing operations previously discussed above. These processing operations include forming a drug-containing polymeric tube using extrusion, radially deforming the formed tube, forming a stent from the deformed tube, crimping the stent, and sterilizing the stent wherein the order of the steps is as presented except that sterilization could be carried out at any earlier point in the process. The various embodiments encompass at of the variations in the processing operations discussed above.

### EXAMPLES

### Example 1

### Biodegradable Polyester Polymer (PLLA) and Paclitaxel Crystalline

PLLA (melting point 150-180 degree C.) with pellet size of approximately 2 mm were first grinded down to less than 500 um with a dry mill and then further grinded down to less than 100 nm using a jet mill. The drug paclitaxel powder were grinded directly in to less than 100 nm using a jet mill. The polymer and drug were mixed in the ratio of 98:2 (by weight) using a speeding mixer at the speed of over 2000 RPM.

### Examples 2

### Paclitaxel-Impregnated Biodegradable Tube Extrusion

200 g of premixed paclitaxel-polymeric composition prepared in example 1 were dried overnight at 45 degree C. The extrusion temperature was set at 160 degree C. with the screw speed of 20 RPM. The extruded paclitaxel-impregnated biodegradable tubes have outside diameter of 1.8 mm, wall thickness of 150 um. The final tube contains, by weight, two percent paclitaxel in at least a portion of crystalline structure. Paclitaxel was evenly dispersed inside the biodegradable polymer.

### Examples 3

### Polymer and Drug Molecular Orientation

The paclitaxel-impregnated tube formed in the example 2 was further deformed using a blow molding technique. In the study the tube was put through a metal mold with an inside diameter of 3.0 mm and pressurized with air at 10 PSI. Heat the metal mold to 60 degrees (10 degree above PLLA's glass transition temperature), hold the tube inside the mold for 30 seconds and then cool the tube quickly to room temperature. Both the drug and polymer's molecules were orientated in both radial and axial direction.

### Example 4

### Laser Cutting the Paclitaxel-Impregnated Biodegradable Tube

The paclitaxel-impregnated biodegradable tube deformed in example 3, were further cut with a femtosecond ultra-pulse laser according to the design specification. FIG. 1 is the image of cut stent with the invented paclitaxel impregnated biodegradable polymer tube.

### Example 5

### HPLC Analysis of Paclitaxel in the Formed Tube

To determine the stability of paclitaxel in the invented drug-impregnated biodegradable stent, 10 mg of drug-polymer composition mixture and one stent were placed in 1 ml extracting solution (50% ethanol and 50% methanol) and continuously shaken at room temperature overnight. The 10 μl extracting solutions were further analyzed by HPLC (HP16 series 1090, Hewlett-Packard Co. Palo Alto, Calif.). The samples were analyzed on a C18-reverse phase column (HP: 4.6×100 mm RP18) using a mobile phase consisting of 0.005% TFA buffer (0.05 ml Trifluoroacetic acid in 1000 ml acetonitrile) delivered at a flow rate of 1.0 mL/min. Paclitaxel peaks from pre-extrusion and stent samples were identical (FIG. 2) indicating that the paclitaxel was not decomposed during extrusion.

### Example 6

### Drug Violability Investigation

To further investigate the violability of drugs encapsulated inside the stent, both tubes, 5 g in each, extracted from the PLLA and PLLA/paclitaxel composition were put into 50 ml drug releasing media (1× cell culture media (MB 752/1, GIBCO) for 4 weeks at 37 degree C. At four weeks, the media was sterilized and further used to culturing the smooth muscle cells (cell type) for one week. After one week, the total cell number in the PLLA/paclitaxel group is significantly less than that in PLLA group indicating that the drug are viable and can effectively inhibit small muscle proliferation.

### Example 7

### Paclitaxel-Impregnated Biodegradable Stent In Vivo Performance and Safety

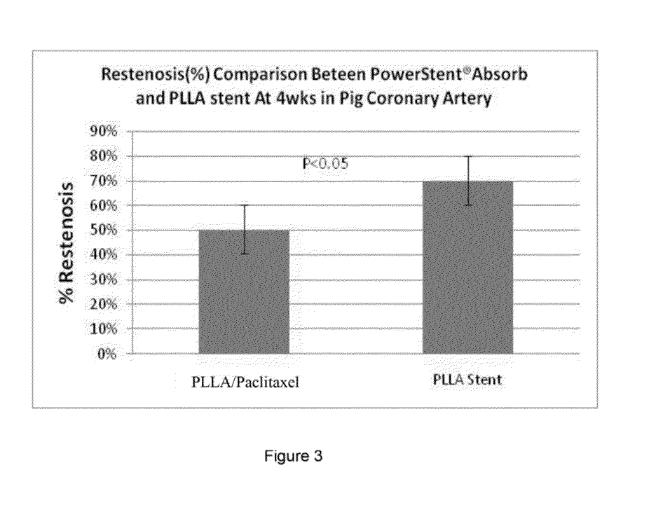

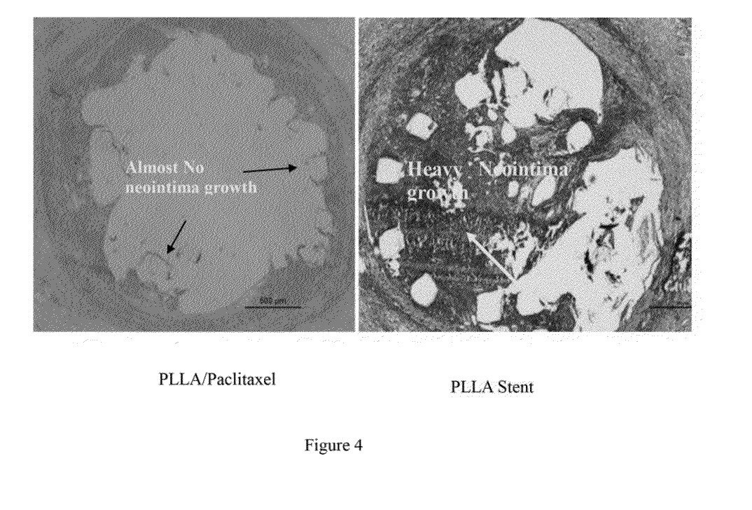

To further investigate the in vivo performance and safety of the invented drug-containing polymeric stent, Six paclitaxel-impregnated stents and six PLLA stents were implanted into pig coronary artery for one month. In the study all twelve stents were successfully implanted into twelve pig's coronary artery without any difficulties. All animal survived one month study period. At one month post implantation, all stented coronary artery remain patency, no any thrombus was found in all twelve animals. The percentage of in-stent restenosis in PLLA/paclitaxel stent group were significantly lower than that in PLLA only group (PLLA/paclitaxel vs. PLLA: 30.5% vs. 71.3%, P<0.005). FIG. 3 depicts the restenosis difference between PLLA/paclitaxel and PLLA stents in pig coronary artery at one month post implantation. FIG. 4 are the histological images shown the difference of neointima and residual arterial lumen area between two groups.

1. A drug-impregnated bioabsorbable stent, the stent, comprising: a stent body fabricated from a biodegradable polyester polymer and at least one therapeutic agent impregnated inside the biodegradable polymer stent body, wherein the at least a port of the therapeutic agent is crystallize. The therapeutic agent is selected from the groups consisting of immunosuppressant agent, anti-neoplastic agent, or/and anti-inflammatory agents.

2. The stent of claim 1, wherein said immunosuppressant agent is selected from the group consisting of sirolimus, zotarolimus, tacrolimus, everolimus, biolimus, pimecrolimus, supralimus, temsirolimus, TAFA 93, invamycin and neuroimmunophilins, and combinations or analogs thereof.

3. The stent of claim 1, wherein said anti-neoplastic agent is selected from the group consisting of paclitaxel, carboplatin, vinorelbine, doxorubicin, gemcitabine, actinomycin-D, cisplatin, camptothecin, 5-fluorouracil, cyclophosphamide, 1-β-D-arabinofuranosylcytosine, and combinations or analogs thereof.

4. A stent of claim 1, wherein aid anti-inflammatory agent is dexamethasone.

5. The stent of claim 1, wherein said biodegradable polyester polymer is selected from the group consisting of PLLA, PDLA, PLA, PGA, and PLGA etc., wherein the selected polymer has a melting point lower than that of impregnated therapeutic agent's as stated in the claim 2, 3, and 4.

6. The stent of claim 1, wherein the ratio between said therapeutic agents and polyester polymer ranges from 1:99 to 30:70, by weight.

7. A method of fabricating an drug-impregnated biodegradable stent the method comprise: selecting compoundable drug-polymer composition, pre-crystallizing both the polymer and therapeutic agent through various nanotechnologies, extruding drug-impregnated polymeric/drug composition through extrusion or injection molding process, orientating both polymer and drug molecular weight through blow molding technique, and finally cutting the stent according to the stent design pattern with ultra-pulse laser technology.

8. The method of claim 7, wherein the therapeutic agent must have a higher melting point than that of the biodegradable polymer of which the therapeutic agent needed to be impregnated.

9. The method of claim 7, wherein the polymer and therapeutic agent are pre-crystallized by various nanotechnologies.

10. The method of claim 7, wherein the drug-impregnated tube or sheet are extruded or injecting molded at the temperature higher than polymer's melting point, but lower than the impregnated drug's melting point.

11. The method of claim 7, wherein the pre-crystallized drug and polymer are premixed and extruded or injection molded.

12. The method of claim 7, wherein the pre-crystallized drug are added to the melted polymer separately through a downstream feeder in an extruder.

13. The method of claim 7, the formed drug-impregnated tube are deformed axially and radially using a blow molding techniques at the temperature of 10 degree C. above the polymer's glass transition point (Tg).

14. The method of claim 7, the deformed drug-impregnated tube is cut with ultra-short pulse laser to designed stent specification.

15. The method of claim 7, further comprise crimping the stent onto a support member prior to sterilizing the stent.
